(19) United States
(12) Patent Application Publication
(10) Pub. No.: US 2002/0049772 A1
(43) Pub. Date: Apr. 25, 2002

(54) Computer program product for genetically characterizing an individual for evaluation using genetic and phenotypic variation over a wide area network

(76) Inventors: Hugh Rienhoff JR., San Carlos, CA (US); Hywell Jones, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/861,081

(22) Filed: May 18, 2001

Related U.S. Application Data
(60) Provisional application No. 60/207,718, filed on May 26, 2000.

(51) Int. Cl. G06F 7/00; G06F 17/00; G06F 15/16; A61B 5/00

(52) U.S. Cl. 707/104.1; 600/300; 709/203

(57) ABSTRACT

The present invention provides a computer program product for separating individuals into subpopulations using a polymorphic profile in a networked environment. The separated groups are subpopulations can be used for clinical studies and treatment studies. The computer program product allows identification of a susceptibility locus in individuals using genetic screening methods to assess their increased risk of certain diseases. In addition, the information can be used to gauge drug responses, study disease susceptibility and to conduct basic research on population genetics.

### CROSS REFERENCE TO RELATED APPLICATIONS

This application claims priority to U.S. Patent Application Nos. 60/207,718, and 60/207,569, both filed on May 26, 2000, the teachings of both applications are hereby incorporated by reference in their entireties for all purposes.

### BACKGROUND OF THE INVENTION

Polymorphism refers to the coexistence of multiple forms of a sequence in a population. Several different types of polymorphisms have been reported. A restriction fragment length polymorphism (RFLP), for example, means a variation in DNA sequence that alters the length of a restriction fragment (see, e.g., Botstein et al., Am. J. Hum. Genet. 32:314-331 (1980)). Short tandem repeats (STRs), as the name implies, are short tandem repeats that consist of tandem di-, tri- and tetra-nucleotide repeat motifs. Such polymorphisms are also sometimes referred to as variable number tandem repeat (VNTR) polymorphisms (see, e.g., U.S. Pat. No. 5,075,217; Armour et al., FEBS Lett. 307:113-115 (1992); and Horn et al., WO 91/14003).

The determination of the presence of polymorphisms, especially mutations, in DNA has become a very important tool for a variety of purposes. Detecting mutations that are known to cause or to predispose persons to disease is one of the more important uses of determining the possible presence of a mutation. One example is the analysis of the gene named BRCA1 that may result in breast cancer if it is mutated (see, Miki et al., Science, 266:66-71, 1994). Several known mutations in the BRCA1 gene have been causally linked with breast cancer. It is now possible to screen women for these known mutations to determine whether they are predisposed to develop breast cancer. Some other uses for determining polymorphisms or mutations are for genotyping and for mutational analysis for positional cloning experiments.

A few different methods are commonly used to analyze DNA for polymorphisms or mutations. The most definitive method is to sequence the DNA to determine the actual base sequence (see, A. M. Maxam and W. Gilbert, Proc. Natl. Acad. Sci. USA 74:560 (1977); Sanger et al., Proc. Natl. Acad. Sci. USA 74:5463 (1977)). Although such a method is the most definitive it is also the most expensive and time-consuming method. Restriction mapping analysis has some use in analyzing DNA for polymorphisms. If one is looking for a known polymorphism at a site which will change the recognition site for a restriction enzyme it is possible simply to digest DNA with this restriction enzyme and analyze the fragments on a gel or with a Southern blot to determine the presence or absence of the polymorphism. This type of analysis is also useful for determining the presence or absence of gross insertions or deletions. Hybridization with allele specific oligonucleotides is yet another method for determining the presence of known polymorphisms. These latter methods require the use of hybridization techniques which are time consuming and costly.

By far the most common form of polymorphisms are those involving single nucleotide variations between individuals of the same species; such polymorphisms are called single nucleotide polymorphisms, or simply SNPs. Some SNPs that occur in protein coding regions give rise to the expression of variant or defective proteins, and thus are potentially the cause of a genetic disease. Even SNPs that occur in non-coding regions can nonetheless result in defective protein expression (e.g., by causing defective splicing). Other SNPs have no phenotypic effects.

Pharmacogenomics describes an area of research of how variations in a patient's DNA can cause pharmaceuticals to respond differently. For instance, differences in the genes that code for cytochrome P-450 affects how patents metabolize drugs differently. This is important because in 1994, for example, two million hospitalizations and more than 100,000 deaths were caused by an adverse drug reaction. Moreover, cataloging genetic variations among SNPs can be used to characterize drug responses. The more SNPs cataloged, the more robust and effective the database. However, collecting and sorting the SNPs becomes a huge undertaking. One way to ease the difficulty in collecting huge amounts of genetic information is via the Internet.

The Internet comprises a vast number of computers and computer networks that are interconnected through communication links. The interconnected computers exchange information using various services, such as electronic mail, Gopher, and the World Wide Web. The WWW service allows a server computer system (i.e., Web server or Web site) to send graphical Web pages of information to a remote client computer system. The remote client computer system can then display the Web pages. Each resource (e.g., computer or Web page) of the WWW is uniquely identifiable by a Uniform Resource Locator (“URL”). To view a specific Web page, a client computer system specifies the URL for that Web page in a request (e.g., a HyperText Transfer Protocol (“HTTP”) request). The request is forwarded to the Web server that supports that Web page. When that Web server receives the request, it sends that Web page to the client computer system. When the client computer system receives that Web page, it typically displays the Web page using a browser. A browser is a special-purpose application program that effects the requesting of Web pages and the displaying of Web pages.

When a user indicates to the browser to display a Web page, the browser sends a request to the server computer system to transfer to the client computer system an HTML document that defines the Web page. When the requested HTML document is received by the client computer system, the browser displays the Web page as defined by the HTML document. The HTML document contains various tags that control the displaying of text, graphics, controls, and other features. The HTML document may contain URLs of other Web pages available on that server computer system or other server computer systems.

In view of the foregoing, what is needed in the art is a computer program product that is capable of generating a polymorphic profile for an individual and thereafter, separating individuals based upon their polymorphic profile. The polymorphic profile can thereafter be used for myriad applications. The present invention fulfills these and other needs.

### SUMMARY OF THE INVENTION

In one embodiment, the present invention provides a computer program product for separating individuals into subpopulations using a polymorphic profile in a networked environment. The separated groups or subpopulations can be used for clinical studies and treatment studies. In a preferred embodiment, the computer program product allows identification of a susceptibility locus in individuals using genetic screening methods to assess an individual's risk of certain diseases. For example, identification of a melanoma susceptibility locus would alert an individual to his/her increased risk of cancer due to sunlight exposure. In addition, the information can be used to gauge drug responses, study disease susceptibility and to conduct basic research on population genetics. In a preferred embodiment, the polymorphic profile data are transferred via a worldwide network of computers such as an internet, the Internet, a combination thereof, and the like.

The computer program product includes code for determining a polymorphic profile of an individual in a population. A polymorphic profile refers to one or more polymorphic forms for which an individual is characterized. A polymorphic form is characterized by identifying which nucleotide(s) is (are) present at a polymorphic site in a nucleic acid sample acquired from an individual. The computer program product also includes code for determining a statistically significant difference between the polymorphic profile for each individual of the population and separating the population into a first subpopulation and a second subpopulation based upon the polymorphic profile. In certain instances, the population is separated using one or more “single nucleotide polymorphism(s)” (SNPs). SNPs occur at polymorphic sites that are occupied by a single nucleotide, the site is a variation between allelic sequences. A single nucleotide polymorphism (SNP) usually arises due to substitution of one nucleotide for another at the polymorphic site.

In another embodiment, the computer code product of the present invention includes code that compares an individual's polymorphic profile with a plurality of polymorphic profiles. In a preferred embodiment, the pluralities of polymorphic profiles contain genetic template profiles. The genetic profiles are stored in a database and the information can be used to gauge drug responses, study disease susceptibility and to conduct basic research on population genetics.

In one aspect, the computer code product includes computer code directed to encoding an individual's polymorphic profile into a transmissible format. The computer code product also includes computer code directed to decoding the encoded polymorphic profile by a processor to permit analysis to be performed. In order to analyze an individual's polymorphic profile the computer code product further includes computer code directed to retrieving data of known polymorphic profiles from a genetic template library and performing an analysis using such data. In addition, the system includes computer code directed to updating the genetic template library with an individual's polymorphic profile.

Numerous benefits are achieved by way of the present invention over conventional techniques. In certain aspects, the computer program product of the present invention can be used to assist in performing clinical trials. In addition, the computer program product of the present invention can be used in pharmacogenomics, wherein an individual nucleic acid variation can be used to ascertain whether the efficacy of a pharmaceutical will be amplified or reduced. As a variation in a single nucleotide in a gene can make an individual susceptible to a particular disease or immune to a specific compound, numerous advantages flow in using the computer program product of the present invention. Using the present invention, it is possible to isolate the genetic causes of disease and subsequently develop better drug treatments tailored to individuals' genetic makeup. Moreover, using the present invention, drug makers are able to ascertain how genes respond to potential pharmaceuticals before they undertake expensive and sometimes dangerous human clinical trials. The computer program product is designed to control for underlying genetic factors that may influence the response to a treatment. The present invention is based, in part, on the insight that controlling, either directly or indirectly, genetic factors that influence a patient's response to treatment can greatly increase the power of the clinical trial or treatment. The computer program product aids in reducing the genetic diversity of the patient population so as to increase the probability of individuals sharing the same alleles at genes involved in response to the treatment. In cases where polymorphisms are known to be associated with or cause differences in response to the treatment, these polymorphisms can be used directly in the design of a clinical trial. Moreover, the nucleic acid sequence and protein variants discernable using the present invention can be used to map any phenotype or trait that can be observed, detected, measured, etc. These include drug responses, disease outcomes, disease susceptibilities, phenotypes, outward appearances, physiological descriptors, etc. In certain aspects, they can be used for identification, paternity, determining ethnicity, etc.

Another advantage of the present invention is that every polymorphic profile that is determined can be added to the electronic genetic profile library thereby continually expanding the repository of knowledge. This approach allows historical data to be kept and retrieved for subsequent use. In addition, with the use of an electronic genetic profile library, data can be easily shared at different physical locations thereby facilitating objective data comparison. For instance, data relating to an individual in one country can be compared to an individual in another country with ease.

These and other aspects and advantages will become more apparent when read with the detailed description and accompanying drawings which follow.

### BRIEF DESCRIPTION OF THE DRAWINGS

FIG. 1 illustrates a representative flow diagram embodying an aspect of the present invention.

FIG. 2 illustrates a representative flow diagram embodying an aspect of the present invention.

FIG. 3 illustrates a representative networked environment for embodying the present invention.

FIG. 4 is an illustration of a representative computer system in an embodiment according to the present invention.

FIG. 5 is an illustration of basic subsystems of the system of FIG. 4.

FIG. 6 illustrates a representative module embodying an aspect of the present invention.

FIG. 7 illustrates a representative flow diagram embodying an aspect of the present invention.

FIG. 8 illustrates a representative flow diagram embodying an aspect of the present invention.

FIG. 9 illustrates a representative flow diagram embodying an aspect of the present invention.

### DETAILED DESCRIPTION OF THE INVENTION AND PREFERRED EMBODIMENTS

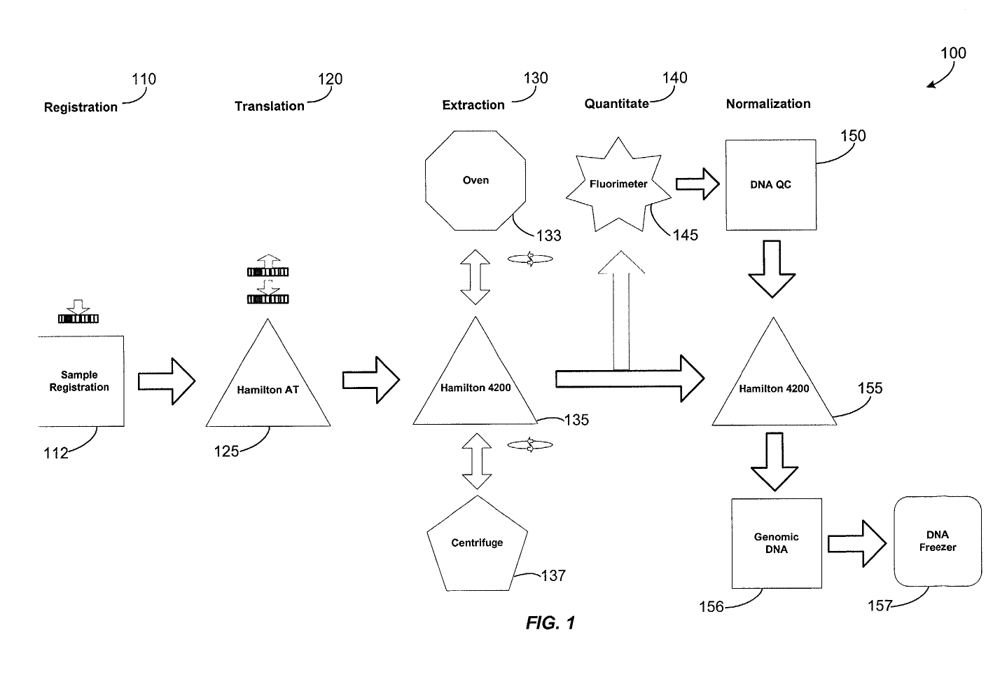

FIG. 1 represents one flow chart 100 that embodies an aspect of the computer program product of present invention. This flow chart is merely an illustration and should not limit the scope of the claims herein. One of ordinary skill in the art will recognize other variations, modifications, and alternatives.

In certain embodiments, the process of determining an individual's polymorphic profile begins with registration 110. Registration 110 embodies the process of receiving a biological sample such as blood, or DNA sample(s), in an individual tube with an external sample ID, either in the form of a barcode or another annotation (handwritten, typed, etc.) attached to the individual tube. This ID is entered into a database and the sample is associated with other information (disease status, drug therapy, phenotype, behavior, family history, etc.) that is received concurrently or has already been received in an electronic format and is entered into a database. Preferably, an internal barcode ID is attached to each sample 112 after the sample is entered into the database. The registration step is typically achieved at a computer workstation with a barcode reader and a barcode printer, and preferably, in a networked environment.

Polymorphic profile termination then proceeds to the next step or the translation step 120. Translation 120 is the step whereby an individual sample, such as blood or DNA, is added to an array of multiple samples, e.g. an array of up to 96 samples, in an 8×12 array. This “plate” of samples is then given a unique ID, whereby any single sample is then associated with both the plate and a particular coordinate within the plate (e.g., well B3). This can be achieved automatically such as by a Hamilton AT2 robot 125 integrated with a barcode reader.

Extraction 130 is typically the next step in polymorphic profile determination. Extraction 130 is the step whereby reagents are added to the blood samples to disrupt the cells, and remove the proteins, sugars, salts, RNA, etc. The resulting product is purified DNA. In certain instances, the sample received in the registration step 110 is already purified DNA, instead of a raw sample (e.g. blood sample), thus the extraction step 130 is omitted. In a preferred embodiment, the extraction step 130 is done automatically using robotic armature such as with a Hamilton 4200 MPH-8 robot 135 for reagent addition steps, an oven for incubation steps 133, and a centrifuge for purification steps 137.

In certain aspects, the next step in determining an individual's polymorphic profile is a quantitation step 140. In this process step, the concentration and purity of DNA for a particular sample is measured. This can be achieved by a variety of methods, including, but not limited to, absorbance at 260 and 280 nm or by fluorescence measurement of DNA-binding dyes. Quantitation can be accomplished using various analytical instrumentation such as a spectrophotometer (for absorbance readings) or a fluorometer 145 (for fluorescence readings).

Following quantitation 140, in certain aspects in the determination of a polymorphic profile, the next step is normalization 150. In the normalization step 150, samples are diluted with a buffer to a standard concentration. After the extraction process, the samples have various concentrations, often between the range of 5-40 ng/μL. Samples will all be normalized to a concentration of approximately 10 ng/μL (±20%), except for samples below a threshold, which will be re-queued to repeat the above process. This step can be done on a Packard Multiprobe robot 155. Thereafter, the genomic DNA sample 156 is placed in a freezer 157 to ensure sample stability. The presence or absence of various alleles predisposing an individual to a disease is determined. Results of these tests and interpretive information are returned to a health care provider for communication to the tested individual. Diagnostic laboratories can perform such diagnoses, or, alternatively, diagnostic kits are manufactured and sold to health care providers or to private individuals for self-diagnosis.

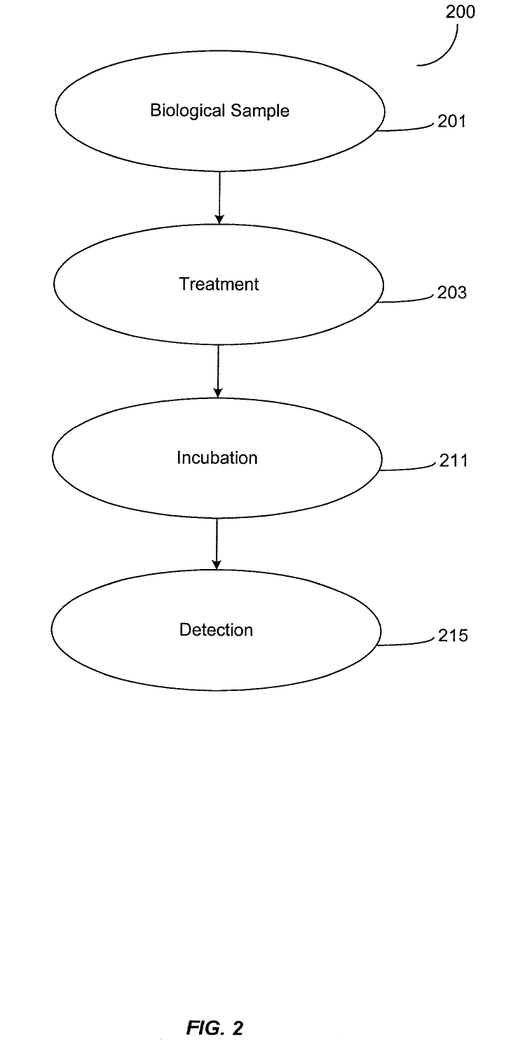

FIG. 2 represents one flow chart 200 that embodies an aspect of the computer program product of present invention. This flow chart is merely an illustration and should not limit the scope of the claims herein. One of ordinary skill in the art will recognize other variations, modifications, and alternatives.

The computer program product of the present invention provides code for screening DNA sequence profiles. Such screening modules employ various methods including but are not limited to, two-step label amplification methodologies that are well known in the art. Both PCR and non-PCR based screening strategies can detect target sequences of individuals with a high level of sensitivity.

In one embodiment, the computer program product of the present invention employs target amplification. In this method, the target nucleic acid sequence is amplified with polymerases. One particularly preferred method using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be sequenced or used as a substrate for DNA probes. The DNA from the blood sample is thereafter compared with others who suffer from a particular disease. In certain instances, the computer program product of the present invention allows better use of existing therapies.

In certain instances, the probes are used to detect the presence of the target sequences in the individual's polymorphic profiles for example, in screening for diabetes susceptibility, the biological sample to be analyzed 201, such as blood or serum, can be treated 203, if desired, to extract the nucleic acids. The sample nucleic acid can be prepared in various ways to facilitate detection of the target sequence; e.g. denaturation, restriction digestion, electrophoresis or dot blotting. The targeted region of the analyte nucleic acid (i.e., the individual's nucleic acid) is usually at least partially single-stranded to form hybrids with the targeting sequence of the probe. If the sequence is naturally single-stranded, denaturation will not be required. However, if the sequence is double-stranded, the sequence may need to be denatured. Denaturation can be carried out by various techniques known in the art.

In a preferred embodiment, analyte nucleic acid and a probe are incubated 211 under conditions that promote stable hybrid formation of the target sequence in the probe with the putative targeted sequence in the analyte. The region of a probe that is used to bind to the analyte can be made completely complementary to the targeted region of human chromosome. High stringency conditions are desirable in order to prevent false positives. However, conditions of high stringency are used only if the probes are complementary to regions of the chromosome that are unique in the genome. The stringency of hybridization is determined by a number of factors during hybridization and during the washing procedure, including temperature, ionic strength, base composition, probe length, and concentration of formamide. These factors are outlined in, for example, Maniatis et al, Molecular cloning: A laboratory manual (Cold Spring Harbor Laboratory, Cold Spring Harbor, N.Y. 1982 and Sambrook et al., Molecular cloning. A laboratory manual, 2nd Ed. (Cold Spring Harbor Laboratory, Cold Spring Harbor, N.Y.) 1989. Under certain circumstances, the formation of higher order hybrids, such as triplexes, quadraplexes, etc., may be desired to provide the means of detecting target sequences.

In one embodiment, detection 215 of the resulting hybrid is accomplished by the use of labeled probes. Alternatively, the probe may be unlabeled, but may be detectable by specific binding with a ligand that is labeled, either directly or indirectly. Suitable labels, and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies and the like. Variations of this basic scheme are known in the art, and include those variations that facilitate separation of the hybrids to be detected from extraneous materials and/or that amplify the signal from the labeled moiety. A number of these variations are reviewed in, e.g., Matthews & Kricka (1988). Anal. Biochem. 169:1.; Landegren et al., Science 242:229 1988; Mittlin, Mittlin (1989). Clinical Chem. 35:1819. 1989; U.S. Pat. No. 4,868,105, and in EPO Publication No. 225,807.

In certain instances, the systems and computer program products of the present invention can use methods for populating a secured database with genotypic and phenotypic data, for example, by using a server coupled with a worldwide network of computers. The method is generally disclosed in U.S. patent application Ser. No. 09/805,813, filed Mar. 13, 2001, and incorporated herein by reference in its entirety for all purposes. The server provides a web site configured to create trust of the web site by users. The method comprises inviting users to submit phenotypic data; inviting users to submit a biological sample; populating the secured database with received phenotypic data; analyzing received biological samples to obtain genetic data; populating the secured database with the genetic data obtained from biological samples; prompting users that previously submitted phenotypic data to submit new phenotypic data; and populating the secured database with received prompted new phenotypic data.

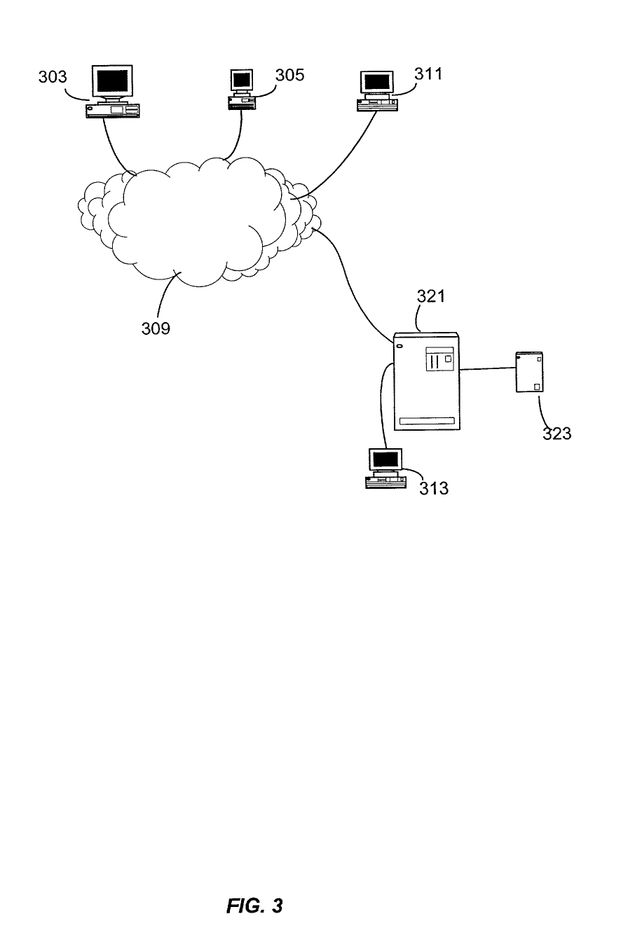

FIG. 3 represents one environment in which the computer program product of present invention can be used. This diagram is merely an illustration and should not limit the scope of the claims herein. One of ordinary skill in the art will recognize other variations, modifications, and alternatives. Environment 300 includes a wide area network 309 such as, for example, the Internet. A plurality of individual clients 303, 305, 311 is connected to network 309. Also connected to the wide area network 309 is an information server 321, with terminal 313 and database 323. Wide area network 309 allows each of computers 303, 305, 311 and 321 to communicate with other computers and each other.

Each of consumer computers 303, 305, 311 can be owned and operated by a different individual. Consumer computers can be configured with many different hardware components and can be made in many dimensions, styles and locations (e.g., laptop, palmtop, pentop, server, workstation and mainframe). For example, computer 303 can be at the home of a first individual, individual computer 305 can be at the home of a second individual, and consumer computer 311 can owned by a third individual, etc. Consumer computer e.g., 303 can include, as one example, conventional desktop personal computers or workstations having the ability to connect to network 309 and being capable of running customized software supporting the service provided by the present invention.

Terminal 313 is connected to server 321. This connection can be by a network such as Ethernet, asynchronous transfer mode, IEEE standard 1553 bus, modem connection, universal serial bus, etc. The communication link need not be a wire but can be infrared, radio wave transmission, etc. Server 321 is coupled to the Internet 309. The Internet is shown symbolically as a cloud or a collection of server routers 309. The connection to server 304 to the Internet is typically by a relatively high bandwidth transmission medium such as a T1 or T3 line.

Internet server 321 and database 323 store information and disseminate it to individual computers e.g. 305 over wide area network 309. The computer program product of the present invention can be used for separating individuals into subpopulations using a polymorphic profile in a networked environment 300. The separated groups are subpopulations can be used for clinical studies and treatment studies. Server 321 connected to wide area network 309 stores physical parameters about a plurality of consumer products on an electronic database 323. The concepts of “client” and “server,” as used in this application and the industry, are very loosely defined and, in fact, are not fixed with respect to machines or software processes executing on the machines. Typically, a server is a machine e.g. 321 or process that is providing information to another machine or process, i.e., the “client,” e.g. 311 that requests the information. In this respect, a computer or process can be acting as a client at one point in time (because it is requesting information) and can be acting as a server at another point in time (because it is providing information). Some computers are consistently referred to as “servers” because they usually act as a repository for a large amount of information that is often requested. For example, a WEB site is often hosted by a server computer with a large storage capacity, high-speed processor and Internet link having the ability to handle many high-bandwidth communication lines.

With respect to the electronic database or electronic genetic profile library 323, it generally contains polymorphic profiles for various genetic templates and other relevant information pertaining to these profiles. The library 323 can be composed of a number of different databases. These databases can be located in one central repository, or alternatively, they can be dispersed among various distinct physical locations. These databases can be categorized and structured in various ways based on the needs and criteria of the database designer. For example, the data can be organized in a database using genetic markers. Possible types of data include SNPs, genetic variations, HLA typing data, identification data, or quality control data. As another example, a first database may contain data relating to various types of HLA class II data collected using the same detection technique under a standardized set of conditions, and a second related database may contain miscellaneous information correlating to data contained in the first database. Methods used to create and organize databases are commonly known in the art, for example, relational database techniques can be used to logically connect these databases.

In one embodiment, as shown in FIG. 3, the databases comprising the genetic profile library 323 or a portion thereof, can be physically located separate from the processor. These databases can reside on remote, distant servers on a local area network or the Internet. Under this arrangement, whenever any data are needed, the processor needs to access the necessary database(s) via a communication channel to retrieve the requisite data for analysis. For example, the processor can access and retrieve data from a remote database via a computer network such as a LAN or the Internet.

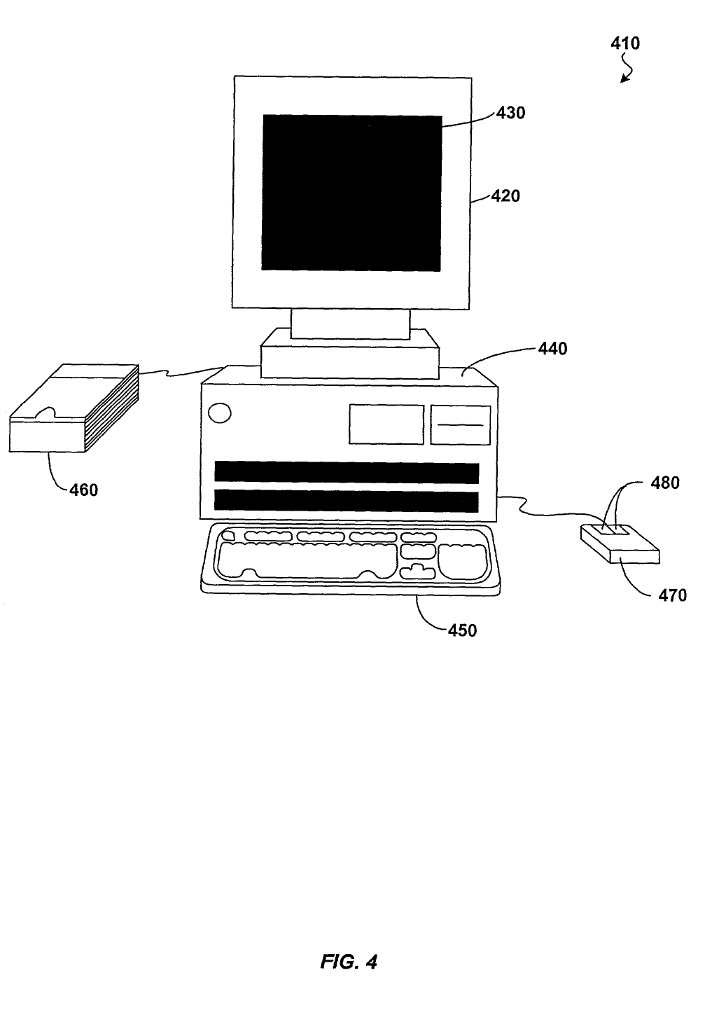

FIG. 4 illustrates a representative system according to a particular embodiment of the present invention. This diagram is merely an illustration and should not limit the scope of the claims herein. One of ordinary skill in the art will recognize other variations, modifications, and alternatives. Embodiments according to the present invention can be implemented in a single application program such as a browser, or can be implemented as multiple programs in a distributed computing environment, such as a workstation, personal computer or a remote terminal in a client server relationship. FIG. 4 shows computer system 410 including display device 460, display screen 430, cabinet 440, keyboard 450, scanner 460 and mouse 470. Mouse 470 and keyboard 450 are representative “user input devices.” Other examples of user input devices are a touch screen, light pen, track ball, data glove and so forth. FIG. 4 is representative of but one type of system for embodying the present invention. It will be readily apparent to one of ordinary skill in the art that many system types and configurations are suitable for use in conjunction with the present invention.

In a preferred embodiment, computer system 410 includes a Pentium® class based computer, running Windows® NT operating system by Microsoft Corporation. However, the apparatus is easily adapted to other operating systems and architectures by those of ordinary skill in the art without departing from the scope of the present invention.

Mouse 470 can have one or more buttons such as buttons 480. Cabinet 440 houses familiar computer components such as disk drives, a processor, storage device, etc. Storage devices include, but are not limited to, disk drives, magnetic tape, solid state memory, bubble memory, etc. Cabinet 440 can include additional hardware such as input/output (I/O) interface cards for connecting computer system 610 to external devices external storage, other computers or additional peripherals.

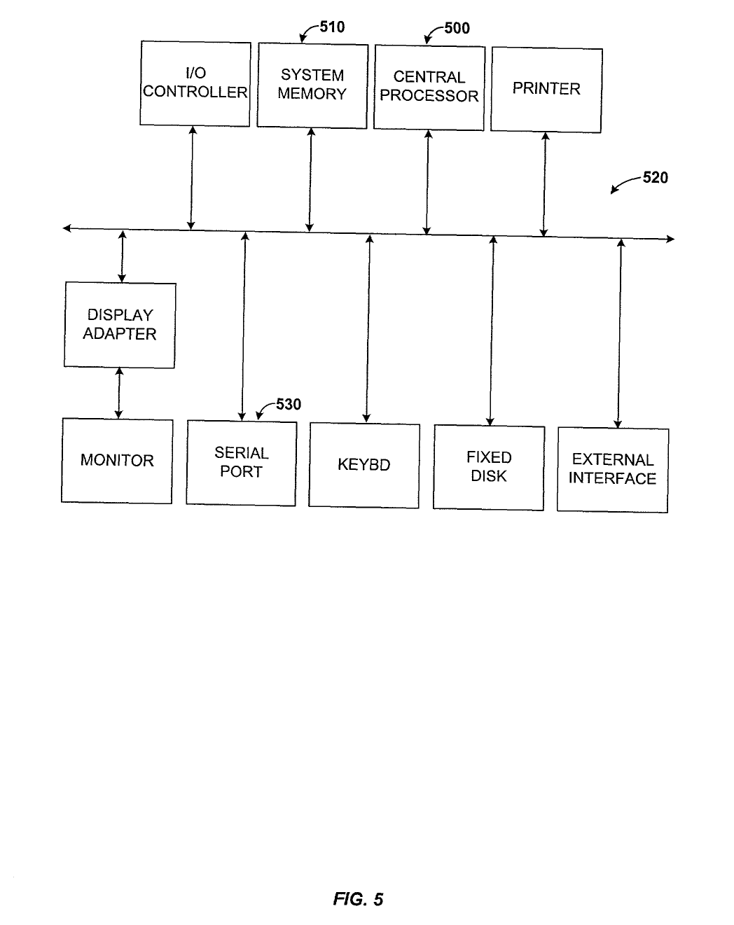

FIG. 5 is an illustration of basic subsystems in computer system 510 of FIG. 4. This diagram is merely an illustration and should not limit the scope of the claims herein. One of ordinary skill in the art will recognize other variations, modifications, and alternatives. In certain embodiments, the subsystems are interconnected via a system bus 520. Additional subsystems such as a printer, keyboard, fixed disk and others are shown. Peripherals and input/output (I/O) devices can be connected to the computer system by any number of means known in the art, such as serial port 530. For example, serial port 530 can be used to connect the computer system to a modem, which in turn connects to a wide area network such as the Internet, a mouse input device, or a scanner. The interconnection via system bus 520 allows central processor 500 to communicate with each subsystem and to control the execution of instructions from system memory 510 or the fixed disk, as well as the exchange of information between subsystems. Other arrangements of subsystems and interconnections are readily achievable by those of ordinary skill in the art. System Memory 510, and the fixed disk are examples of tangible media for storage of computer programs, other types of tangible media include floppy disks, removable hard disks, optical storage media such as CD-ROMS and bar codes, and semiconductor memories such as flash memory, read-only-memories (ROM), and battery backed memory. The computer program product of the present invention allows individual to track the progress of studies, and disease development online.

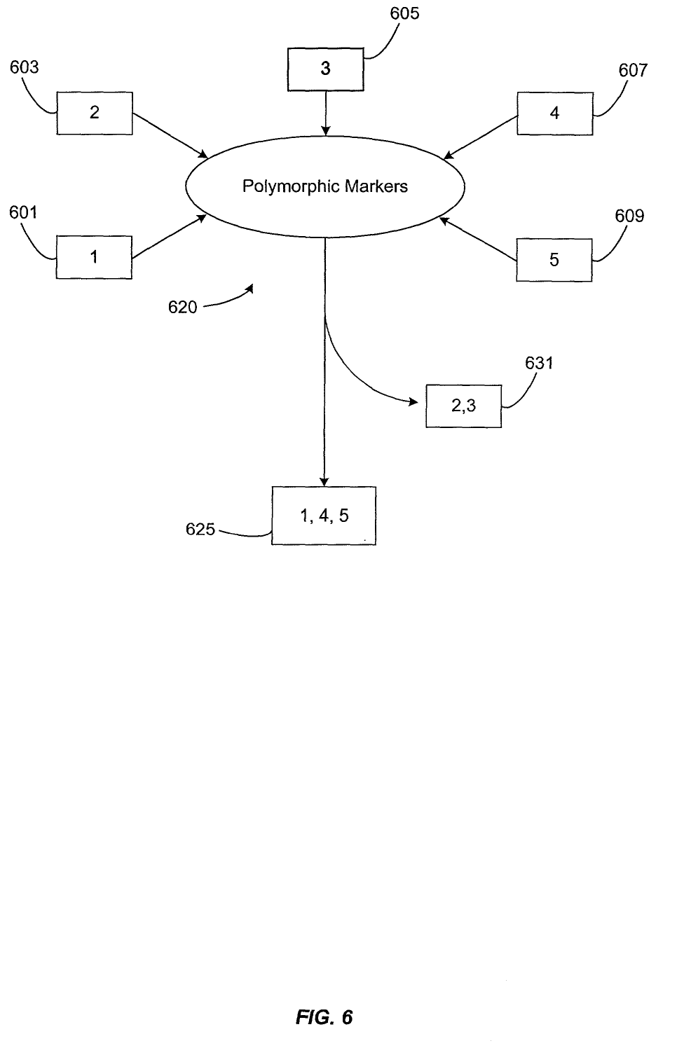

FIG. 6 illustrates a representative system module 600 according to a particular embodiment of the present invention. This diagram is merely an illustration and should not limit the scope of the claims herein. One of ordinary skill in the art will recognize other variations, modifications, and alternatives.

In certain embodiments, the present invention provides a computer program product that facilitates increasing the homogeneity of a select population and thereby the selective enrollment of patients. One approach is to control for potentially confounding factors by increasing the homogeneity of the population. In the context of genetics, a set of polymorphic markers 620 can be examined in a large group of subjects 601, 603, 605, 607, 609 and those with similar polymorphic profiles enrolled in the treatment study. Incorporating genetic factors (represented by the polymorphic profile) into the inclusion group 625 or exclusion group 631 of a treatment study allows an experimenter to reduce the variance in response due to underlying genetic factors.

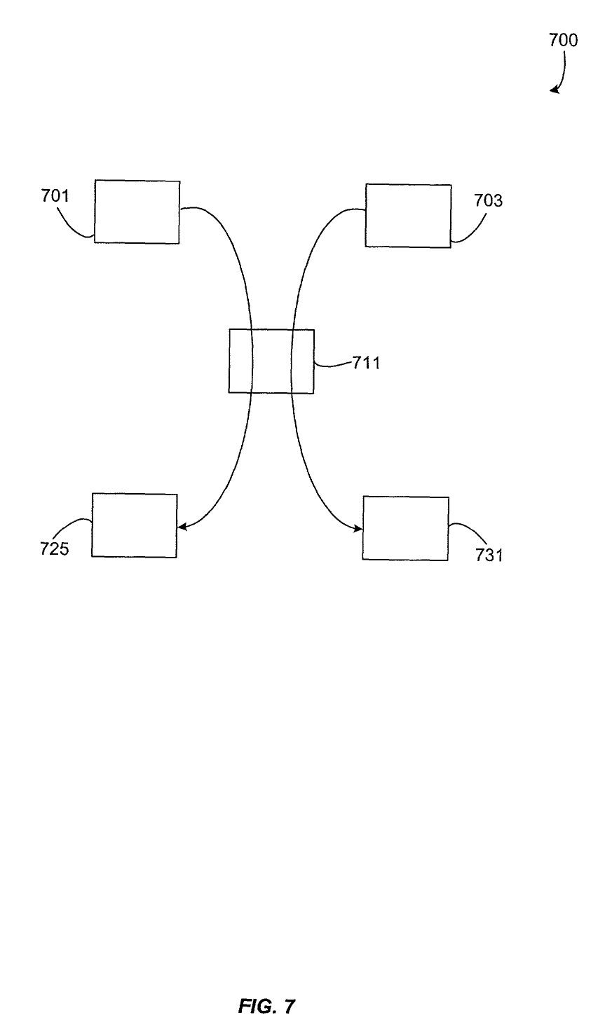

FIG. 7 illustrates a representative computer code flow diagram 700 according to a particular embodiment of the present invention. This diagram is merely an illustration and should not limit the scope of the claims herein. One of ordinary skill in the art will recognize other variations, modifications, and alternatives.

In one embodiment, the computer code product of the present invention provides code that functions to divide a patient population into genetically homogenous subsets. In this approach, individuals 701, 703 are categorized into subsets depending on how similar the polymorphic profiles 711 are to one another. Within each subset, subjects are randomly allocated into treatment 725 or control subpopulations 731, as they are in a standard clinical trial for example. This method of dividing the subjects creates subsets that are genetically more homogenous than a random sample of the same size. This design is equivalent to conducting several small, independent treatment studies, each of which contains patients that have similar polymorphic profiles than expected by chance. Many environmental variables can be manifestations of underlying genetic factors. By examining genetic polymorphisms directly, it is possible not only to reduce variance due to genetic factors that are not directly observable, but also to improve the stratification based on environmental factors that are acting as surrogates for the underlying genetics factors that control them. As used herein, stratification refers to the division of the sample into subsets that are more similar than expected by chance for a given factor.

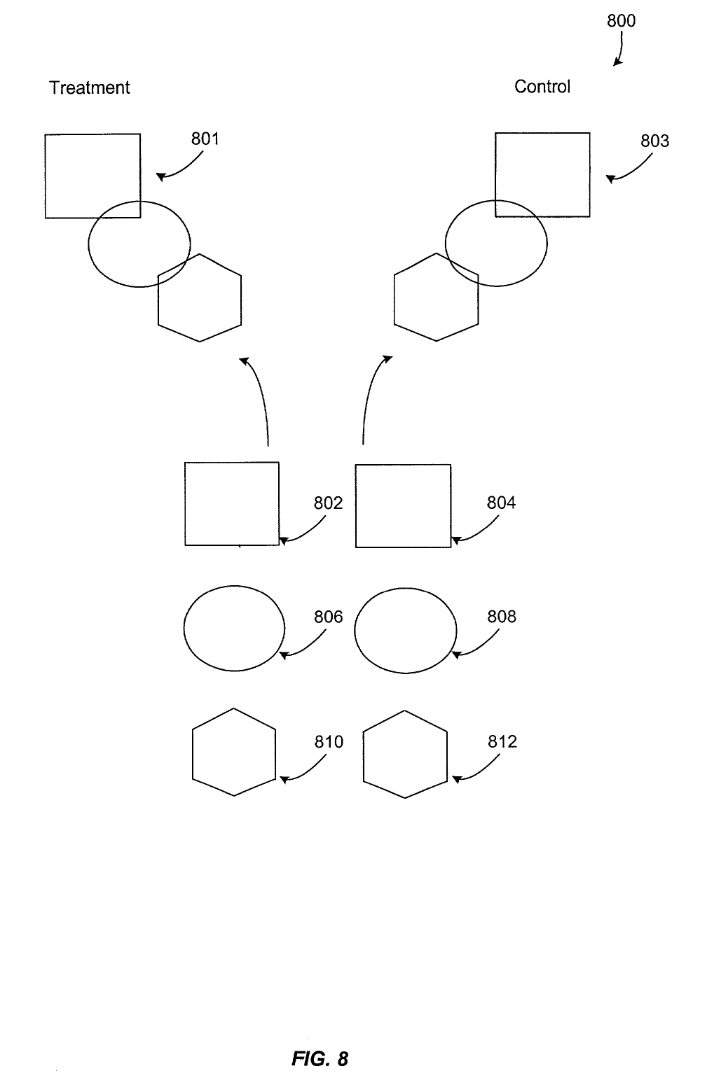

FIG. 8 illustrates a representative computer program product flow diagram 800 according to a particular embodiment of the present invention. This diagram is merely an illustration and should not limit the scope of the claims herein. One of ordinary skill in the art will recognize other variations, modifications, and alternatives.

In one embodiment, the computer program product of the present invention provides computer code for matching patients by their polymorphic profiles. In this approach, the subjects in the treatment 801 and control groups 803 are matched. That is, pairs of individuals with similar polymorphic profiles 802, 804; 806, 808; and 810, 812 are sought and one is allocated to the treatment group 801 while the other is placed in the control group 803. In this way, the difference in response of each pair can be examined where the pairs have been matched for their underlying genetics. Matching on the basis of genetic factors can control new, previously unknown causes of variance due to genetic factors and also provide greater discriminatory power when matching by environmental factors that have an underlying genetic cause.

Moreover, when one or more known polymorphisms is known to be associated with the response to treatment, these can be used directly to allocate patients into treatment and control groups. In the simplest case where a subject's polymorphic profile indicates whether or not they will respond to the treatment, this information can be used as an exclusion/inclusion criterion at the time of enrollment, thus reducing the sample size needed to observe a given level of response. Alternatively, all subjects can be enrolled in the treatment study with the treatment non-randomly assigned. For example, those known to be non-responders by their polymorphic profile can be treated according to a control procedure (e.g., administered a placebo), while those who deemed responders from their polymorphic profile can be given the treatment procedure (e.g., administered a drug). This maximizes the difference in response between treatment and control groups. Conversely, non-responders can be given the treatment and responders the treatment. In this scenario, the minimum difference between treated and untreated subjects can be evaluated.

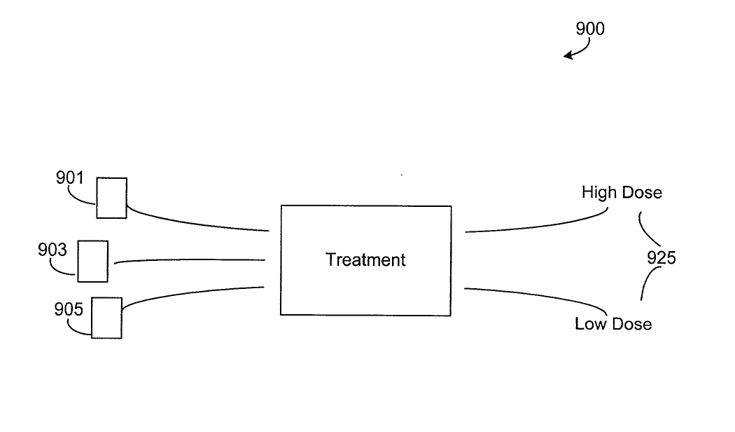

FIG. 9 illustrates a representative a computer program product flow diagram 900 according to a particular embodiment of the present invention. This diagram is merely an illustration and should not limit the scope of the claims herein. One of ordinary skill in the art will recognize other variations, modifications, and alternatives.

When one or more known polymorphisms is known to be associated with response to treatment, this information may be used to allocate the most appropriate dose to subjects enrolled in a treatment study such as a clinical trial. The polymorphic profiles 901, 903, and 905 of individuals can determine the degree of response of individuals to the treatment 910. In this way, it is be possible to allocate different doses to different patient depending on their polymorphic profiles. For example, if a treatment potentially has side effects, it will be desirable to administer the minimum efficacious dose. This can vary for subjects with different polymorphic profiles.

The present invention also provides a re-analysis code that can be used after the completion of a treatment study such as a clinical trial, wherein data obtained from such a treatment study are re-analyzed on subsets of the treated and control populations selected for similarity of a polymorphic profile to each other. The reanalysis of data is carried out on subsets of individuals sharing a similar polymorphic profile and indicates whether the treatment reaches statistical significance on individuals having that profile. If the profile contains one or more polymorphic forms associated in some way with the biological condition of interest (e.g., disease), the treatment may reach statistical significance on the subpopulations when it does not on the initial treatment populations. If the profile does not contain such polymorphic DNA forms, then the re-analysis of data also shows a lack of statistical significance. At this point, a further re-analysis is performed in which further subpopulations of individuals from treated and control populations are selected for similarity to a second polymorphic profile. Because the individuals have already been characterized for polymorphic profile, the second re-analysis can be performed without further experimental work in a highly automated and iterative fashion. Again, the second analysis indicates whether the treatment reaches statistical significance on the individuals having similarity to the polymorphic profile by which subpopulations are selected in the second analysis.

Subsequent rounds of analysis can be performed according to the same principles without further experimental work. A suitably programmed computer can perform thousand, millions or billions of cycles of analysis in which different subpopulations of individuals are selected based on similarity to different polymorphic profiles. Performing multiple tests typically requires a re-evaluation of the p-value at which a result is declared to be statistically significant to control the rate of false positive results. If after exhaustive analysis, statistical significance is not reached for any polymorphic profile, one can conclude with increased confidence that the treatment procedure (e.g., administration of a drug) being tested is unlikely to be effective in any significant portion of the population, and that further research is not justified. If, however, statistical significance is reached for a particular polymorphic DNA profile, at least two conclusions follow. First, in the case of a clinical trial on a drug that the drug is effective in at least a portion of the population, and further development of the drug may well be justified. Second, one knows the portion of the general population in which the drug is effective, this portion being defined by a polymorphic profile. This profile can be used as a diagnostic to identify patients appropriate for treatment when the decision to treat or a choice of treatments is made.

In certain embodiments, the computer program product of the present invention can be used for detecting predisposition to cancer at the MTS gene as disclosed in U.S. Pat. No. 5,989,815, which issued to Skolnick, et al., on Nov. 23, 1999, and the MTS2 gene as disclosed in U.S. Pat. No. 5,994,095, which issued to Kamb on Nov. 30, 1999. As disclosed therein, somatic mutations in the Multiple Tumor Suppressor (MTS) gene can be used for the diagnosis and prognosis of human cancer. Moreover, germ line mutations in the MTS gene can also be used in the diagnosis of predisposition to melanoma, leukemia, astrocytoma, glioblastoma, lymphoma, glioma, Hodgkin's lymphoma, CLL, and cancers of the pancreas, breast, thyroid, ovary, uterus, testis, kidney, stomach and rectum.

In another embodiment, the computer program product of the present invention can be used for detecting predisposition to cancer as disclosed in U.S. Pat. No. 5,989,885, which issued to Teng, et al., on Nov. 23, 1999. As disclosed therein, specific mutations of map kinase 4 (MKK4) in human tumor cell lines, identify it as a tumor suppressor in various types of cancer. The gene can be used in the diagnosis and prognosis of human cancer. Specific polymorphism such as mutations in the MKK4 gene, is associated with breast, pancreatic, colorectal and testicular cancers.

In still another embodiment, the computer program product can be used for detecting the predisposition for cancer using the (BRCA2) gene, some mutant alleles of which cause susceptibility to cancer, in particular breast cancer. In certain aspects, diagnostic methods for the predisposition to cancer using the BRCA2 gene are disclosed in U.S. Pat. No. 6,033,857, which issued to Tavtigian, et al. on Mar. 7, 2000. As disclosed therein, germline mutations in the BRCA2 gene can be used in the diagnosis of predisposition to breast cancer. Moreover, somatic mutations in the BRCA2 gene can be used in human breast cancer detection and the prognosis of human breast cancer.

In still yet another embodiment, the computer program product can be used for detecting the predisposition to hypertension. For instance, U.S. Pat. No. 5,998,145, which issued to Lalouel, et al. on Dec. 7, 1999, discloses a method to determine predisposition to hypertension. As disclosed therein, there is an association of the molecular variant G-6A of the angiotensinogen gene with human hypertension. The determination of this association enables the screening of persons to identify those who have a predisposition to high blood pressure.

As an example of a method of the invention, a clinical trial can be carried out as follows:

### 1. Identification and Choice of Polymorphisms

A set of polymorphisms is identified that allow the division of the patient cohort into sub-groups. These polymorphisms may be known to be involved in the test parameter (e.g., the phenotype or endpoint) that is to be measured or can be chosen at random. (In the latter case, the genetic sub-groups may show identical results with respect to the phenotype of interest. This implies the method of grouping does not decrease the variance in the endpoint and the population can be re-analyzed as a whole. Thus, stratification by using genetic data does not have a deleterious effect on the experiment or trial, even in cases where it does not influence the outcome).

### 2. Genotyping of the Cohort

Some or all of the markers are genotyped in the entire cohort of patients enrolled in the clinical trial. These data are then used either as inclusion/exclusion criteria (see 3a below) or to divide the cohort into subgroups (see 3b below).

### 3a. Inclusion/Exclusion of Patients Using Genetic Information

If some or all of the polymorphisms are known to influence the test parameter that is to be measured, it may be appropriate to exclude individuals when it is known, a priori, they will present a particular phenotype or endpoint. In the context of a clinical trial, this can represent excluding those individuals who, by information gained from the set of polymorphisms examined, will not respond to the therapy.

### 3b. Division of the Clinical Trial Into Subgroups

A metric is used to determine the genetic similarity of patients in the cohort. This information is used to divide the population into subgroups that have greater genetic similarity than might be expected by chance. That is, the subgroups are genetically more homogenous than a random subset of the same size.

The precise method of measuring similarity will depend on the number and type of markers used. In the simplest case, the number of markers at which two individuals have the same alleles can be used to determine similarity. Many other more complex metrics can be employed that, for example, giving extra weight to markers known to be particularly informative or that influence the test parameter of interest.

By altering the method of determining genetic similarity, an experimenter can control the number of subgroups that need to be formed. For N individuals, this can range from 1 (the entire population) to N (each individual is in a separate subgroup). Practical as well as scientific reasons are considered in determining how many subgroups are optimal for a given experiment or trial. With the methods of the invention, groups can be merged at a later time.

### 4. Allocation of Treatment Within the Genetic Subgroups

When the patients have been grouped into genetic subgroups based on information from the set of polymorphism described in 1, several strategies are available for conducting a treatment study such as a clinical trial.

One method is to randomize the treatment and placebo within each subgroup. This is similar to treating each subgroup as a separate experiment or clinical trial. Results of each subgroup may be analyzed separately or may be pooled and then analyzed.

Alternatively, treatment can be non-randomly allocated within the subgroups. This may be appropriate, for example, when the polymorphisms are known to be associated with the outcome or endpoint of interest. For example, in the context of a clinical trial, if there are only two subgroups and one of the subgroups is known to contain high responders and the other low responders to a treatment, allocating the treatment to the first group and the placebo to the second group maximizes the difference between response for treated and untreated individuals. Conversely, allocating the placebo to the first group and the treatment to the second group shows the minimum difference between treated and untreated individuals. Which of these approaches is most appropriate depends on the exact objective of the experiment or clinical trial.

### 5. Use of Information From One Experiment in the Design of Subsequent Studies

The utility of stratifying by using a set of genetic polymorphisms can be re-assessed through successive experiments of clinical trials. Uninformative polymorphisms can be dropped and new polymorphisms added to increase the usefulness of the set as a whole. Use of these polymorphisms in subsequent treatment studies or a clinical trial leads to greater reproducibility of results and the need for enrolling fewer subjects in replication studies.

By identifying and correlating polymorphisms to a particular effect of a drug, and thus reducing the variance due to genetic factors, a clinician can devise clinical trials that involve fewer subjects, decrease the confidence intervals, or increase the precision or discriminatory power of a given trial. The clinician can decide which of these three aspects of trial design or analysis to change while keeping the other two constant.

In addition to altering the statistic of variance which in turn can affect subject number, precision or power of a study, using analysis of polymorphic markers in a clinical trial population in a manner as disclosed herein permits, upon analysis, the identification of subsets of polymorphic markers that may correlate with either a salubrious response, unresponsiveness or excessive response to a treatment, an unwanted or toxic response to a treatment, and may identify by virtue of unresponsiveness, a clinical subset of patients that define a “different” disease. In short, a post facto genetic analysis correlated with a specific clinical phenotype such as drug responsiveness or unresponsiveness can reveal different etiologic mechanisms for the disease being treated. This is especially likely in the case of ethnic differences among patients where each ethnic group has a distinctive response to a treatment. Finally, analysis of phenotypic markers can provide insight into genetic diversity of the subjects being treated allowing the clinician to alter enrollment in a drug trial to accommodate more or less genetic diversity as is scientifically prudent.

In yet another embodiment, the systems and computerprogram products of the present invention provide a method for populating a database for further medical characterization through a worldwide network of computers. The methods are generally disclosed in U.S. patent application Ser. No. 09/805,619, filed Mar. 13, 2001, and incorporated herein by reference. The method comprises populating a database with a plurality of user health information from a plurality of users, the user health information including genetic data and phenotypic data for a user; and wherein the database is populated at least in part through browsing activities of the plurality of users on the world wide network of computers.

While the invention has been described with reference to certain illustrated embodiments this description is not intended to be construed in a limiting sense. For example, the computer platform used to implement the above embodiments include 586 class based computers, Power PC based computers, Digital ALPHA based computers, SunMicrosystems SPARC computers, etc.; computer operating systems may include WINDOWS NT, DOS, MacOs, UNIX, VMS, etc.; programming languages may include C, C++, Pascal, an object-oriented language, etc.

Various modifications of the illustrated embodiments as well as other embodiments of the invention will become apparent to those persons skilled in the art upon reference to this description. In addition, a number of the above processes can be separated or combined into hardware, software, or both and the various embodiments described should not be limiting.

All publications, patents and patent applications mentioned in this specification are herein incorporated by reference into the specification in their entirety for all purposes. Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

What is claimed is:

1. A computer program product for separating individuals into subpopulations using a polymorphic profile in a networked environment, said networked environment comprising at least one client connected to at least one server by a network, said computer program product comprising:
code for determining a polymorphic profile of an individual in a population;
code for determining a statistically significant difference between said polymorphic profile for each individual of said population and separating said population into a first subpopulation and a second subpopulation based upon said polymorphic profile; and
a computer readable storage medium for holding said codes.

2. The system of claim 1, wherein said code for determining a statistically significant difference is executed at said server.

3. The computer program product of claim 2, wherein said first subpopulation and said second subpopulation are used in a clinical trial.

4. The computer program product of claim 1, wherein said first subpopulation and second subpopulation are used in a pharmacogenomic study.

5. The computer program product of claim 1, wherein said first subpopulation and second subpopulation are used in an epidemiological study.

6. The computer program product of claim 1, wherein said first subpopulation and second subpopulation are used in a predisposition study.

7. The computer program product of claim 1, wherein said first subpopulation and second subpopulation are used in a susceptibility study.

8. The computer program product of claim 1, wherein said first subpopulation and second subpopulation are used in forensic studies.

9. The computer program product of claim 1, wherein said first subpopulation and second subpopulation are used in paternity determination.

10. The computer program product of claim 1, wherein said network is the Internet.

11. A computer program product for analyzing genetic information of an individual in a networked environment, said networked environment comprising at least one client connected to at least one server by a network, said computer program product comprising:
code for imputing an information object from said at least one client corresponding to a polymorphic profile of an individual;
code for receiving said information object at said at least one server, said at least one server having a storage device for storing a plurality of template profiles;
code for analyzing said polymorphic profile of an individual compared to said plurality of template profiles to generate a match profile; and
a computer readable storage medium for holding said codes.

12. The computer program product according to claim 11, wherein said network comprises the Internet.

13. The computer program product according to claim 11, further comprising code directed to retrieving polymorphic profiles from a genetic library.

14. The computer program product according to claim 11, further comprising a code directed to displaying results of execution of said code directed to analyzing on a web page.

15. The computer program product according to claim 11, wherein imputing an information object from said client is conducted via wireless communications.

16. The computer program product according to claim 15, wherein said wireless communications are implemented using communications technologies selected from the group consisting of infrared technology, satellite technology, microwave technology and radio wave technology.

17. A system including memory and computer codes for analyzing genetic data pertaining to an individual's polymorphic profile, said system comprising:
code directed to transmitting data pertaining to known template profiles from a genetic library to a first location via a computer network;
code directed to receiving said data pertaining to known template profiles at said first location; and
code directed to analyzing said data pertaining to said individual's polymorphic profile using said received data pertaining to known template profiles at said first location and generating an analysis result.

18. The system according to claim 17, further comprising:
code directed to receiving input from a user; and
code directed to using said input during execution of said code directed to analyzing.
